(19) United States
(12) Patent Application Publication
(10) Pub. No.: US 2014/0030254 A1
(43) Pub. Date: Jan. 30, 2014

(54) SIGNAL PATHWAY ALTERATIONS AND DRUG TARGET ELEVATIONS IN PRIMARY METACHRONOUS METASTATIC COLORECTAL CANCER COMPARED TO NON-METASTATIC DISEASE

(71) Applicants: Emanuel F. Petricoin, III, Gainesville, VA (US); Lance A. Liotta, Bethesda, MD (US); Mariaelena Pierobon, Manassas, VA (US); Alessandra Silvestri, Capriva del Friuli (IT)

(72) Inventors: Emanuel F. Petricoin, III, Gainesville, VA (US); Lance A. Liotta, Bethesda, MD (US); Mariaelena Pierobon, Manassas, VA (US); Alessandra Silvestri, Capriva del Friuli (IT)

(21) Appl. No.: 13/851,644

(22) Filed: Mar. 27, 2013

Related U.S. Application Data
(63) Continuation of application No. 13/057,409, filed on Apr. 14, 2011, now abandoned.
(60) Provisional application No. 61/086,275, filed on Aug. 5, 2008.

(51) Int. Cl. G01N 33/574

(52) U.S. Cl. CPC G01N 33/57419
USPC 424/133.1; 506/9; 435/7.23; 435/7.4; 506/18; 514/406; 514/473; 514/291; 514/266.4; 514/266.24; 514/234.5; 424/138.1; 514/386; 702/19

(57) ABSTRACT

The present invention relates to the identification and diagnostic use of biomarkers in primary colorectal cancer tumors whose activation level are predictive of the likelihood of the onset of metastatic disease. These biomarkers may be used to determine the suitability of a patient for aggressive and/or targeted treatments. Kits and compositions of the invention are also provided.

### CROSS-REFERENCE TO RELATED PATENT APPLICATIONS

This application is a continuation application under 35 U.S.C. §§111(a) and 120 of U.S. patent application Ser. No. 13/057,409, filed Apr. 14, 2011, which is a national phase application of, and claims priority to and the benefit of, International Patent Application No. PCT/US2009/052901, filed Aug. 5, 2009, which claims priority to and the benefit of U.S. Provisional Patent Application No. 61/086,275, filed Aug. 5, 2008.

### BACKGROUND OF THE INVENTION

Colorectal cancer (CRC) is the most frequent malignancy of the digestive tract and one of the most common solid organ cancers in developed countries. The estimated rate of CRC in the U.S. in 2008 is 148,810, and the expected death rate is 50,640. Development of metastases is the main cause of death among CRC patients, as approximately one third of CRC patients initially staged M0-N0 die from tumor recurrence. Because the survival rate of CRC patients is strictly related to the presence of these metastases, prognostic biomarkers that can identify distant or occult metastases can lead to better diagnoses, as well as better treatment options.

Cellular proteins, particularly those associated with cell signaling, can be used as biomarkers for cancer that better predict the cancer progression, as well as treatment outcome. Traditionally, gene expression analysis was used to determine if a particular gene was overexpressed in a cancer; however, quantification of gene expression is not as determinative of treatment outcome or responsiveness as the activation level of the protein expressed by that gene.

For example, c-erbB2 (Her-2/neu) is a protein in the epidermal growth factor (EGF) signaling pathway that is overexpressed in approximately 30% of breast cancers as well as some prostate and bladder cancers. This overexpression was believed to cause the aberrant activation of the protein, and therefore, therapeutics that target this protein, such as HERCEPTIN®, were administered to patients that overexpressed c-erbB2. However, as reported in International Patent Application PCT/US2009/49903, it has been found that the activation level of c-erbB2, not overexpression of the gene, is a better prognostic marker and predictor of HERCEPTIN responsiveness.

Similarly, treatment of CRC with therapeutics that target a specific signal protein or pathway may be enhanced if the activation state of the target is known. For example, inhibitors of the Cox2/EGFR pathway, ckit inhibitors such as imatinib mesylate (GLEEVAC®) and other pathways may be used to treat CRC in which these signal proteins are activated. Alternatively, simply identifying which patients are likely to develop metastatic CRC can be treatment more aggressively with traditional therapeutic agents. Therefore, profiles of the activation levels of proteins involved in protein signaling provide a more accurate prognostic signature than traditional gene expression analyses.

### SUMMARY OF THE INVENTION

The present invention provides a method for predicting if a subject with colorectal cancer is likely to develop one or more metastases or has occult metastasis, comprising the steps of:

(A) preparing a sample from the primary tumor;

(B) measuring the activation level of one or more target proteins is the sample selected from the group consisting of:

a. mTOR,

b. 4EBP1,

c. Adducin,

d. cKit,

e. cRaf,

f. Stat3,

g. HistoneH3,

h. IRS,

i. PDGFR beta,

j. Pyk2,

k. S6 Ribosomal Protein,

l. Stat5,

m. VEGFR,

n. C1-Caspase9,

o. C1-NOTCH,

p. Cox2,

q. EGFR,

r. pBAD,

s. pcAb1, and

t. pPKC alpha; and

(C) comparing the activation level of (B) to positive and/or negative reference standards to determine if the target protein is activated;

wherein the activation level of (B) is determined by measuring the phosphorylation of the target protein, the total amount of the target protein or the proteolytic cleavage products of a target protein; and wherein the activation of one or more target proteins indicates that the patient is likely to likely to develop metastases.

In a further embodiment, the present method further comprises

(D) calculating a pathway signature score by
(i) summing the activation levels of the target proteins of (B); and
(ii) dividing the sum of (i) by the activation level of a target protein associated with non-metastases, and

(E) determining a cutpoint of the pathway signature score of (D) such that none of the subjects with samples having a pathway signature score below the cutpoint develop metastases.

In a further embodiment, the target protein associated with non-metastases of (ii) is pPKC alpha.

In one embodiment, the subject is a human patient and the colorectal cancer is likely to metastasize to the patient's liver.

In one embodiment, step (A) of the present method further comprises:

(i) isolating epithelial cells from the sample;

(ii) lysing the epithelial cells to form a lysate; and

(iii) contacting the lysate with a detectable label to detect the target protein.

In a further embodiment, step (i) of the method comprises using laser capture microdissection on the sample.

In one embodiment, step (B) of the method comprises using an assay selected from the group consisting of immunoassays, colorimetric assays, assays based on fluorescent readouts, histochemical assays, mass spectroscopy, and Western blot.

In a further embodiment, the lysate is distributed onto a reverse phase microarray and then analyzed by an immunoassay.

In one embodiment, step (B) of the method comprises measuring the level of phosphorylation of one or more of the following proteins:

a. pCox2,

b. pBAD,

c. pcKit,

d. pPDGFRb,

e. pEGFR,

f. pS6 Ribosomal protein,

g. pmTOR,

h. pAb1,

i. pAdducin,

j. pBcl2,

k. pcRaf,

l. pEGFR,

m. C1-NOTCH, and

n. PKC alpha.

In alternative embodiments, the activation levels of at least two, at least three, at least four, at least five or at least six of the target proteins are measured. In alternative embodiments, the target proteins of (B) are at least one of mTOR, cKit, PDGFR beta, EGFR, Cox2 and VEGFR. In further embodiments, the target proteins of (B) are at least one of mTOR (S2481), cKit (Y703), PDGFR beta (Y751), EGFR (Y1148), EGFR (Y1173), Cox2 and VEGFR (Y951).

The present invention also provides a method for treating, delaying or preventing metastasis in a human patient with colorectal cancer comprising the steps of:

(A) preparing a sample from the primary tumor;

(B) measuring the activation level of one or more target proteins in the sample selected from the group consisting of:

a. mTOR,

b. 4EBP1,

c. Adducin,

d. cKit,

e. cRaf,

f. Stat3,

g. HistoneH3,

h. IRS,

i. PDGFR beta,

j. Pyk2,

k. S6 Ribosomal Protein,

l. Stat5,

m. VEGFR,

n. C1-Caspase9,

o. C1-NOTCH,

p. Cox2,

q. EGFR,

r. pBAD,

s. pcAb1, and

t. PKC alpha; and

(C) comparing the activation level of (B) to positive and/or negative reference standards to determine if the target protein is activated; and

(D) treating the patient with a targeted or aggressive therapy if the activation of one or more target proteins of (C) indicates that the patient is likely to develop metastases,

wherein the activation level of (B) is determined by measuring the phosphorylation of the target protein, the total amount of the target protein or the proteolytic cleavage products of a target protein.

In one embodiment, step (B) of the above method comprises measuring the level of phosphorylation of one or more of the following proteins:

a. pCox2,

b. pBAD,

c. pcKit,

d. pPDGFRb,

e. pEGFR,

f. pS6 Ribosomal protein,

g. pmTOR,

h. pAb1,

i. pAdducin,

j. pBcl2,

k. pcRaf,

l. pEGFR,

m. C1-NOTCH, and

n. PKC alpha.

In a further embodiment, the treatment of (D) comprises treating the patient with an effective amount of a therapeutic agent that targets at least one of the activated target proteins. In a further embodiment, the therapeutic agent is one or more agents selected from the group consisting of CELECOXIB, REFECOXIB, TORISEL, TARCEVA, LAPATINIB, IRESSA, ERBITUX, BEVTUZIMAB, AVASTIN, GLEEVEC, DASATINIB, and SUTENT. In a further embodiment, the method further comprises administering a conventional chemotherapeutic agent to the patient.

The present invention also provides kits for determining the prognosis of a patient having CRC from a sample of a primary CRC tumor comprising:

(i) one or more reagents for determining the activation level of at least one of
a. mTOR,
b. 4EBP1,
c. Adducin,
d. cKit,
e. cRaf,
f. Stat3,
g. HistoneH3,
h. IRS,
i. PDGFR beta,
j. Pyk2,
k. S6 Ribosomal Protein,
l. Stat5,
m. VEGFR,
n. C1-Caspase9,
o. C1-NOTCH,
p. Cox2,
q. EGFR,
r. pBAD,
s. pcAb1, and
t. PKC alpha; and

(ii) instructions for performing the assay.

In one embodiment of the kit, the subject is a human patient.

In alternative embodiments, the kit contains reagents for assaying the phosphorylation state of at least one, two, three or all of mTOR, cKit, PDGFR beta, EGFR, Cox2 and VEGFR. In a further embodiment the kit comprises reagents for assaying the phosphorylation state of at least one, two, three or all of the following: mTOR (S2481), cKit (Y703), PDGFR beta (Y751), EGFR (Y1148), EGFR (Y1173) and VEGFR (Y951).

In one embodiment the reagents of the kit are selected from the group consisting of antibodies, aptamers, and ligands specific for the protein or proteins being assayed. In a further embodiment, the reagents are antibodies. In a further embodiment, the reagents are monoclonal antibodies. The kit may also further comprise packaging materials.

The present invention provides a pharmaceutical composition, comprising a therapeutically effective amount of:

(a) a targeted therapeutic agent of at least two target proteins selected from the group consisting of mTOR, cKit, PDGFR, EGFR, Cox2 and VEGFR; and

(b) a pharmaceutically acceptable carrier.

In a further embodiment, the composition may also comprise a therapeutically effective amount of carboxyamido imidazole, CELECOXIB, REFECOXIB, TORISEL, TARCEVA, LAPATINIB, IRESSA, ERBITUX, BEVTUZIMAB, AVASTIN, GLEEVEC, DASATINIB, and SUTENT.

### BRIEF DESCRIPTION OF THE DRAWINGS

FIG. 1 is the pathway signature score for the target proteins identified in heat maps with the best correlation with metastases, which are listed in Table 5. One heat map showed the unsupervised clustering analysis of the indicated target proteins in eight primary CRC tumors from patients that developed metastatic metachronous tumors compared to eight primary CRC tumors that did not progress to metastatic metachronous disease. The activated target proteins are listed in Table 1. A second heat map showed the indicated target proteins in 22 primary CRC tumors in patients with lymph node infiltration versus 22 primary CRC tumors without lymph node infiltration. The activated target proteins are listed in Table 2. A third heat map showed the indicated target proteins in the eight primary CRC tumors from patients that developed metastatic metachronous tumors compared to the fifty tumors that did not (14 lymph node positive, 36 non-metastatic). The relative intensity values of these highly specific biomarkers were summed, then divided by the relative intensity value of pPKC alpha (PKCa), which is a marker for non-metastatic CRC tumors. As shown in the scatter plot, this ratio is very sensitive to detecting CRC tumors with occult metastases (squares) as compared to non-metastatic tumors with or without lymph node infiltration (up and down pointing triangles, respectively). A cutpoint value below which no metastatic tumors are found was determined. Here, the cutpoint value is 15 (dashed line), which give 8/8 true positives, 11/50 false positives, 39/39 true negatives and 0/8 false negatives.

FIG. 2 is a Kaplan-Meir survival plot of the CRC patients using the PKCa-based ratio cutpoint determined in FIG. 1. The upper line is those patients that were below the cutpoint, and the upper line is those patients above the cutpoint. The y-axis is percent survival.

### DETAILED DESCRIPTION OF THE PREFERRED EMBODIMENTS

The present invention provides methods for identifying biomarkers for determining the prognosis of colorectal cancer (CRC), particularly CRC likely to develop into metastatic CRC by determining the activation level of target signaling proteins in the primary tumor. The present invention also provides methods for determining the responsiveness of the CRC to a treatment based on the on activation level of target signaling protein in a primary tumor. These methods are more accurate and reliable than current methods for characterizing CRC tumors.

Without being bound by the theory, it is believed that patients that develop metastatic CRC are likely to have occult metastases even at the time of M0 or stage 1 diagnosis. Such occult metastases may develop further even if the primary tumor is surgically removed. Regardless of the possible correlation with occult metastases, it has been found surprisingly that the signaling profile of the primary tumor can more accurately predict the likelihood of metastatic progression even in the absence of traditional prognostic indicators.

The singular forms “a,” “an,” and “the” refer to one or more, unless the context clearly indicates otherwise.

The terms “subject” and “patient” are used interchangeably, and are meant to refer to any mammal, including humans, that has, or is at risk of developing CRC. The subject or patient is typically human, however, other suitable subjects or patients include, but are not limited to, laboratory animals, such as mouse, rat, rabbit, or guinea pig, farm animals and domestic animals or pets. Non-human primates are also included. The present methods can be used at any stage of CRC. For example, the methods can be used with subjects having early stage cancer; subjects having late-stage cancer and subjects in remittance from cancer, including recurring cancer; and subjects having active cancer, including active recurring cancer.

The term “colorectal cancer” or CRC refers to any proliferative disease of the colon or rectum, such as colorectal carcinoma, and may be at any stage, such as stage 0 through 4. Metastatic CRC, also known as aggressive CRC, may include invasiveness through the full thickness of the bowel wall, spread to local or regional lymph nodes, or spread to distant sites such as the liver or lungs. This latter type is also called metachronous metastatic CRC and represents the most deadly form of CRC.

A “sample” may be any suitable cell or tissue that can be assayed to determine the activation status of the target signaling proteins. Suitable samples may include, e.g., tumor biopsies which can be excised from the tissue using any suitable method in the art. In particular, samples of a particular cell type, whether normal or diseased, may be micro-dissected using laser-capture micro-dissection techniques, as described in U.S. Pat. Nos. 6,251,516 and 6,251,467, as well as in U.S. application Ser. No. 10/798,799, each of which is hereby incorporated by reference in its entirety. Briefly, LCM allows for isolation of pure populations or subpopulations of the desired cell type, such as a diseased cell population or a normal cell population, or both even from the same tissue sample. The cells of interest can be identified, e.g., morphology, in situ immunohistochemistry, or fluorescent microscopy. By combining microscopy-based cell identification techniques with laser activation of the polymeric substrate to which the tissue sample is applied, very precise extraction of the desired cells is possible. These cells can then be further characterized, such as for additional markers, or lysed for use in the present invention. Such precision allows for extremely accurate characterization of the desired cells.

“Reference standards” refer to cells or cell lysates from cells or cell lines, such as tumor cell lines, with known disease or signaling protein characteristics, such as a known activation level and activation status. For example, a lysate derived from cells known to have a target signaling protein that is activated and with a high level of activation status may be used to determine if a diseased cell also has an activated target signaling protein. Reference standards may also refer to a series of cells or cell lysates that do not have activated target signaling protein, and the target can be added or “spiked” into the cell or cell lysate in known quantities. Reference standards may also be normal or non-pathological cells of the same cell type as the disease cells, or cells with a known disease state.

The “activation status” of the target protein refers to a qualitative determination of whether the target protein is activated. To determine the activation status, the activation level, or change in the target signaling protein is quantitated and compared to reference standard.

In some embodiments, the target protein is activated if it is phosphorylated. Other forms of alterations in the target protein that indicate activation include glycosylation, farnesylation, dephosphorylation, translocation, proteolytic cleavage and association with another molecule. Alternatively, the total amount of the target protein may be altered, for example, increased, when activated. Any detectable change in the target signaling protein may be used to determining the activation level and activation status in the present invention.

Once the activation level is measured, usually as a measure of intensity relative to the reference standard (i.e., relative intensity) a pathway signature score is generated. This score sums the relative intensity of the target proteins that are prognostic for disease progression (e.g., metastases), then divided by the relative intensity of a target that is associated with nonprogression. For example, target proteins whose activation status is highly correlated with metastases may be used. In one embodiment, the target proteins identified in Table 4 can be used. Their relative intensity values were summed, then divided by the relative intensity of pPKC alpha, which is associated with non-metastatic primary CRC tumors.

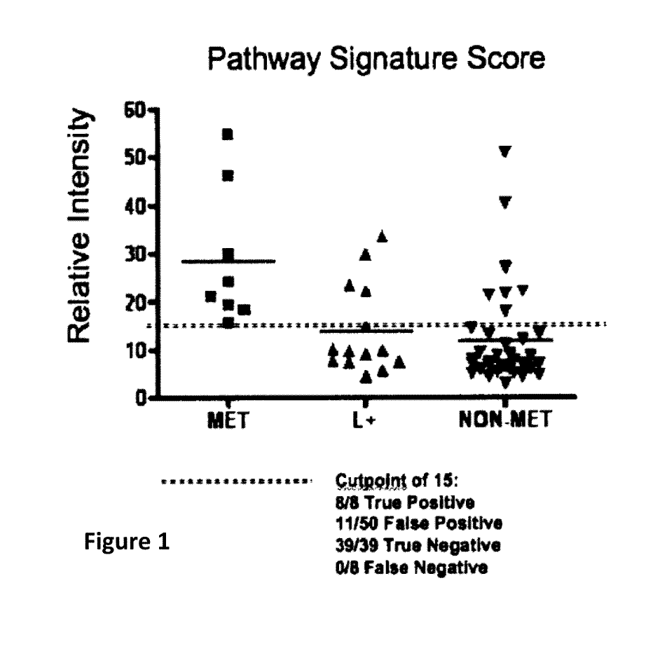

The term “cut-point” refers to the value of the pathway signature score below which no false negatives are detective. In other words, none of the primary CRC tumors with pathway signature scores below the cutpoint are from patients that develop metastatic disease. False positives above the cutpoint, e.g., those tumors that are indicated as likely associated with metastatic disease but do not develop metastatses, are tolerated so as to not miss any tumors associated with metastatic disease. See FIG. 1 for a graphical representation of a cut point.

Such cutpoints will vary from assay to assay based on comparison to reference standards, and may be generated based on receiver operating characteristic (ROC) curves. The ROC method is a graphical plot of the sensitivity vs. (1-specificity) for a binary classifier system as its discrimination threshold is varied. See, e.g., Cleophas et al. Curr. Clin. Pharmacol. (2008) 3:70-76, which is hereby incorporated by reference. For the present analysis, ROC curves with maximum sensitivity is preferred. For more examples of cut-point determination, reference is made to International Appl. No. PCT/US09/049,903, which is hereby incorporated by reference.

The present invention provides methods for determining if the form of CRC is “responsive” to the therapeutic agent. If the sample displays an activation status of signaling proteins that are associated with a known responsiveness, then it is “responsive” and may be treated with that therapeutic agent for enhanced effectiveness. Alternatively, the CRC form is determined to be responsive if the activation status or activation level of a target signaling protein in the diseased cell is changed upon administration of the therapeutic agent as compared to the activation status or activation level of the target signaling protein prior to administration. For example, the target signaling protein may be activated in the sample prior to administration of the therapeutic agent and inactivated after administration. Alternatively, the target signaling protein may be inactivated in the sample prior to administration of the therapeutic agent and activated after administration. In each of these alternatives, the form of the CRC is considered “responsive” to the therapeutic agent. If no change is observed before and after administration, the CRC form is considered “nonresponsive”.

In another embodiment, comparison of treated to untreated CRC samples can also be made serially or in parallel using two populations of the same cells such that the effects of the therapeutic agent can be determined. For example, CRC samples to which the therapeutic agent has been administered can be compared directly to samples to which no therapeutic agent has been administered. The use of reference standards may be used to normalize the measurements to account for experimental variability. In this way, the present invention can discover therapeutic agents that were previously unappreciated for their effectiveness in treating CRC.

As used herein, the term “target protein” is any protein whose activation level is associated with or prognostic for a type of CRC disease progression, such as predictive of the development of metastases. A target protein may be a signaling protein. A “signaling protein” refers to a protein associated with a cellular signaling pathway that is activated or inactivated with CRC. Suitable target proteins are discussed in more detail below.

Examples of signaling pathways that may be associated with CRC include the integrin pathway, the focal adhesion signaling pathway, the Akt/mTOR signaling pathway, the IL-6R pathway, growth factor pathways, chemokine receptor signal pathways, cell-cycle signaling pathways, stress signal pathways, apoptosis signaling pathways, Taulbeta signaling pathways, pro-inflammatory pathways, differentiation signaling pathways, T-cell receptor pathways, death-receptor signaling pathways, survival signaling pathways, MAPK signaling pathways, p38 MAPK signaling pathways, G-coupled receptor signaling pathways, SAPKfJNK signaling pathways, insulin receptor signaling pathways, Wnt signaling pathways, B-cell antigen signaling pathways, cKit signaling pathways, and Jak/Stat signaling pathways. Any pathway or signaling protein associated with CRC may be used in the present invention.

Measuring the activation level may be measured using any available method including protein microarray analysis, immunohistochemistry, antibody microarray analysis, bead capture, western blotting, enzyme-linked immunosorbent assay (ELISA), suspension bead array, or any semi-quantitative immunoassay based methodology. In particular embodiments reverse phase protein microarray analysis is used. In more particular embodiments, reverse phase protein microarray analysis is used to detect phosphorylated signaling protein and/or the total amounts of the signaling proteins regardless of their phosphorylation state.

Briefly, a protein microarray is an assay format that utilizes a substrate for simultaneously testing multiple samples as well as for testing multiple target proteins in the same assay. The microarray format is not limited to particular embodiments but can comprise any arrangement and substrate that serves to provide a plurality of individual samples for testing. For example, in some embodiments, the microarray comprises a flat substrate with rows and columns of individual spots, each spot comprising a sample, while in other embodiments, the microarray comprises a flat substrate with a plurality of depressions, for example, a 96-well plate, in which each depression contains one sample. Examples of typical microarray substrates include nitrocellulose, derivatized glass slides, and 3-dimensional substrates such as hydrogels. Examples of nitrocellulose-coated glass slides include FAST slides (Schleicher & Schuell BioSciences, Keene, N.H.), which have protein binding capacities of 75-150 ug/cm2 in a volume of 0.3-2.0 nl/spot. Nitrocellulose-coated glass slides are particularly useful, as a variety of detection methods can be used with this substrate, including chromogenic, fluorometric and luminescent detection methods.

The number of samples that can be deposited onto a microarray substrate can vary. The size of the substrate can often determine how many samples are located on the substrate. In some embodiments, the protein microarray comprises around 100 spots; in other embodiments, the protein microarray may comprise around 1,000 spots or around 10,000 spots. In yet other embodiments, the microarray comprises from about 1 to about 10,000 spots, about 50 to about 10,000 spots, or about 500 to about 10,000 spots. In some embodiments, the microarray comprises less than about 100,000 spots.

The sample volume which is deposited on each spot and used to form each spot on the microarray can also vary. The volume can depend on diameter of the pin (contact printing), the inherent qualities of the pin hydrophobicity and the method of supplying the sample. In some embodiments, the amount of sample deposited/printed can range from less than about 1 picoliter to about 100 nanoliters.

Samples can be placed or loaded onto the substrate using any one of a number of mechanisms known in the art (see Schena, “Microarray biochip technology” Eaton Pub., Natick Mass., 2000, incorporated herein by reference in its entirety). For example, in some embodiments, the samples are printed onto the microarray using a printer. The printing technique can be contact or non-contact printing, and can be automated.

Protein microarray formats can fall into two major classes, the Forward Phase Array (FPA) and the Reverse Phase Array (RPMA), depending on whether the analyte is capture from solution phase or bound to solid substrate. Forward Phase Arrays immobilize a bait molecule, such as a antibody designed to capture a specific analyte within a mixture of test sample proteins. In FPAs, the capture molecule specific for the analyte is immobilized on a substrate. The capture molecule is then exposed to the sample, binding the analyte in the sample and immobilizing the analyte onto the substrate. The bound analyte can then be detected using a detectable label. The label can bind to the analyte directly, or can be attached to a secondary “sandwich” antibody that is specific for the analyte. The capture molecule can be any molecule that has specificity for an analyte and includes, but is not limited to, peptides, proteins, antibodies or fragments thereof, oligomers, DNA, RNA, and PNA. In some embodiments, the capture molecule is an antibody or fragment thereof specific for the analyte.

Reverse Phase Arrays (RPMAs) immobilize the test sample analytes on a solid substrate. In RPMAs, the sample is placed directly on the substrate, allowing analyte in the sample to bind directly to the substrate. A detection molecule specific for the analyte is then exposed to the substrate, allowing an analyte-detection molecule complex to form. The detection molecule can comprise a detectable label to indicate the presence of the analyte. Alternatively, a secondary molecule specific for the detection molecule and comprising a detectable label can be provided, allowing for an analyte-detection molecule-labeled secondary molecule complex to form. RPMAs are highly sensitive and do not require a large amount of sample. The high sensitivity exhibited by RPMAs is due in part to the detection molecule, which can be conjugated to a detectable label, and is also due in part to the fact that the signal from the label can be amplified independently from the immobilized analyte. For example, RPMAs can use tryamide amplification which generates high number of florescent signal on each spot, or florescent signals that are near-IR wavelength, which is outside the emission spectra for nitrocellulose. Amplification chemistries that are available take advantage of methods developed for highly sensitive commercial clinical immunoassays (see, for example, King et al., J. Pathol. 183: 237-241 (1997)). Using commercially available automated equipment, RPMAs can also exhibit excellent “within run” and “between run” analytical precision. RPMAs do not require direct labeling of the sample analyte and do not utilize a two-site antibody sandwich. Therefore, there is no experimental variability introduced due to labeling yield, efficiency or epitope masking

In a preferred embodiment, RPMA is used to measure activation levels of target proteins associated with CRC. The detection molecule and secondary molecule can be any molecule with specificity for CRC target proteins and capture molecule, respectively. Examples of detection and secondary molecules include, but are not limited to, peptides, proteins, antibodies or fragments thereof, oligomers, DNA, RNA, and PNA. In those embodiments in which both a detection molecule and a secondary molecule are present, the detection and secondary molecules can be the same type of molecule, e.g., a protein, or can be different types of molecules, e.g., the detection molecule can be DNA, and the secondary molecule can be an antibody. In some embodiments, both the detection molecule and the secondary molecule are antibodies or fragments thereof.

In some embodiments, the detection or capture molecule, and, if present, the secondary molecule, are both antibodies or fragments thereof. The antibody or fragment thereof that functions as the capture or detection molecule is specific for the target protein, specific for either the activated form of the target protein being measured, or specific for total target protein, regardless of activation state. The antibody or fragment thereof that functions as the secondary molecule, if present, is typically specific for the detection antibody. Antibodies suitable for detecting both activated and total target protein can be chosen readily by those skilled in the art. See, for example, U.S. patent application Ser. No. 10/798,799, “Combinatorial Therapy for Protein Signaling Diseases,” filed Mar. 10, 2004, the entire contents of which is herein incorporated by reference. Suitable antibodies can also be obtained commercially, for example, from Cell Signaling, Inc. (Danvers, Mass.) and BD Biosciences (San Jose, Calif.). In both FRAs and RPMAs, the capture molecule, the detection antibody, and the secondary molecule, if present, can comprise a detectable label. For example, the capture molecule, the detection molecule, or the secondary molecule, if present, can be conjugated to a detectable label.

Examples of suitable detectable labels include, but are not limited to, fluorescent, radioactive, luminescent and colorimetric labels. Methods and techniques for detecting each type of label are well known in the art.

For fluorescent labels, the labels can have excitation and/or emission spectra in the infrared, near-infrared, visible, or ultra-violet wavelengths. A wide range of fluorescent probes are commercially available (see, e.g., Invitrogen Corporation, Carlsbad, Calif., LI-COR Biosciences, Lincoln Nebr.). Examples of suitable fluorescent probes include, but are not limited to, phycoerythrin or other phycobilliproteins such as allophycocyanin, lanthanide-based dyes, and phthalocyanine dyes. In addition, methods and reagents for coupling fluorescent probes to proteins, including antibodies, are well known in the art (see, for example, technical handbooks from Invitrogen Corporation (Carlsbad, Calif.) and Pierce (Thermo Fisher Scientific, Inc., Rockford, Ill.).

Suitable radioactive labels include those containing the isotopes C14, P32, and S35. Examples of suitable luminescent labels include quantum dots, 1,2-dioxetanes, and luminal. Examples of suitable colorimetric labels include DAB. Methods for using each of these labels and their corresponding detection systems are known to the artisan skilled in the art.

In some embodiments, the signal from the detectable label can be amplified. Amplification is helpful for achieving sensitivity adequate for analysis of relatively low abundance proteins. Amplification of the label signal can be achieved by enzymatic cleavage of colorimetric, luminescent or fluorescent substrates, by utilizing avidin/biotin signal amplification systems known in the art, or by taking advantage of the polymerase chain reaction by coupling nucleic acids to protein for detection. For example, amplification chemistries can take advantage of methods developed for highly sensitive commercial clinical immunoassays. See, for example, King et al., J. Pathol. 183:237-241 (1997). Coupling the capture molecule with highly sensitive tyramide-based avidin/biotin signal amplification systems can also yield detection sensitivities down to fewer than 1,000-5,000 molecules/spot. In a particular embodiment, a biopsy of 10,000 cells can yield 100 RPMA microarrays, and each array can be probed with a different antibody.

The measurements obtained for the target signaling protein in each sample can be “normalized” to total protein in the sample using methods known in the art, such that the detected activation level of the target signaling protein is independent of the amount or concentration of the sample spotted on the array. For example, each lysate is measured for the targeted signaling protein as well as total protein as measured by SYPRO Ruby Red protein stain (Molecular Probes, Eugene Oreg.), obtained by staining a different slide with the total protein stain.

The present invention may be used to identify candidates for targeted and/or aggressive treatment by identifying subjects with CRC that is likely to metastasize before such metastases is normally detectable. With early intervention, progression from non-metastatic CRC to metastatic CRC may be prevented or delayed.

Any therapeutic agent that affects a signaling protein to cure, treat, amelioriate, prevent, delay or diagnose CRC may be used in the present invention. For example, the therapeutic agent may be a small molecule compound, a protein, such as an antibody, ligand, aptamer, enzyme or a cytokine, or a nucleic acid, such as a small interfering RNA (siRNA). In one embodiment, the therapeutic agent targets one or more signaling pathways. In a further embodiment, the therapeutic agent targets one or more target protein. In a further embodiment, the therapeutic agent is CELECOXIB, REFECOXIB,TORISEL, TARCEVA, LAPATINIB, IRESSA, ERBITUX, BEVTUZIMAB, AVASTIN, GLEEVEC, DASATINIB, and/or SUTENT. Additional examples of therapeutic agents can be found in WO 2008/057305, which is incorporated herein in its entirety. Alternatively, a therapeutic agent that targets a particular signaling pathway or target protein may be combined with traditional chemotherapeutics or other treatments used to treat CRC.

An “aggressive treatment” is a treatment that is used for CRC that has metastasized or is believed to be likely to metastasized. Such aggressive treatment may include a targeted therapeutic as described above or a traditional or chemotherapeutic treatment that is used for metastatic CRC, such as those listed in WO 2008/053705. The targeted therapeutic may be combined with the traditional treatment.

Accordingly, the present method may be used to identify novel therapeutic agents for the treatment, prevention, amelioration or diagnosis of CRC. The test therapeutic agent may be tested using cells derived from one or more CRC disease types. After administration, the activation of one, or more preferably, more than one target protein is measured so as to determine which signaling pathways are affected by the test agent.

The present invention also provides a pharmaceutical composition comprising an effective amount of inhibitor or stimulator of a target protein to cure, treat, ameliorate, prevent or delay the progression of non-metastatic CRC to metastatic CRC. This inhibitor or stimulator may be a therapeutic agent as described above. The pharmaceutical composition may comprise a pharmaceutically acceptable carrier or excipients. For information regarding such carriers and excipients, see, e.g., Remington's Pharmaceutical Sciences, 18^{th} ed., Mack Publishing Company (1990) or later editions. One of skill in the art would readily be able to develop compositions suitable for administration to a subject, as well as determine the dose of the therapeutic agent necessary to cure, treat, amelioriate, prevent or delay the progression of non-metastatic CRC to metastatic CRC.

Kits for use in the methods of the present invention are also contemplated. Such kits may comprise one or more reagents for assaying the activation level of one or more target proteins in a primary tumor from a subject having CRC. These kits may include a lysis buffer for the sample, antibodies for detecting the activation level of the target protein, and, optionally, reagents for determining the total protein level of a sample. Such kits typically also include instructions for carrying out the method.

The following examples are for illustrative purposes only and do not limit the invention.

### EXAMPLES

### Example 1

To determine if signaling pathway activation could be detected in primary CRC tumors and correlated with metastastic disease progression, samples of primary CRC tumors resected from 58 M0 patients were analyzed. Patients were followed for two to five years for the development of secondary lesions. Of the 58 patients, 36 did not develop secondary lesions during follow up (no metastases), 14 patients were lymph node positive at the time of diagnosis (M0 Stage III, LNM) and eight developed distal metachronous metastases (MM, occult metastases) within one to three years of diagnosis and surgery.

Each sample was surgically collected and immediately snap frozen. Pure populations of tumor epithelial cells from 8 μm sections of the frozen tumor samples were stained with hematoxylin and isolated by laser capture microdissection (LCM). Microdissected cells were suspended in lysis buffer at a concentration of 100 cells/μl and heated at 100° C. for 8 minutes to lyse the cells.

Reverse phase microarray (RPMA) analysis was used to measure the activation levels of the target proteins. Arrays were printed with spots of the samples on sets of 100 slides using the 2470 Aushon arrayer (Aushon BioSystems Ins., Billerica, Mass.). Each sample was printed in duplicate and in two-point dilution curves, with an estimated cellular equivalent of 20 cells in the neat (undiluted) spot and 5 cells in the 1:4 dilution spot. Negative and positive controls consisting of cell lysates from cells that were unstimulated or stimulated with either pervandate, calyculin A or etoposide were also printed.

The arrays were blocked and stained with Sypro Ruby Protein Blot Stain (Molecular Probes, Eugene, Oreg.) to normalize the protein amounts for each spot/signal. The arrays were then stained with 75 antibodies that detect the total target protein or the activated (cleaved or phosphorylated) target protein. These antibodies are provided as Table 1.

TABLE 1
Antibodies

Caspase-3, cleaved (D175)
Caspase-9, cleaved (D315)
CD44
CD133
c-ErbB2/HER2
Cox2
EGFR
EGFR L858R Mutant
Estrogen Rec alpha (62A3)
Phospho-4E-BP1 (S65)
Phospho-Adducin (S662)
Phospho-Akt (S473)
Phospho-Akt (T308)
Phospho-ASK1 (S83)
Phospho-Bad (S112)
Phospho-BAD (S136)
Phospho-Bcl-2 (S70)
Phospho-c-Abl (T735)
Phospho-c-Abl (Y245)
Phospho-Catenin(beta) (T41/S45)
Phospho-Chk-2 (S33/35)
Phospho-c-Kit (Y703)
Phospho-c-Kit (Y719)
Phospho-c-Raf (S338) (56A6)
Phospho-CREB (S133)
Phospho-EGFR (Y1068)
Phospho-EGFR (Y1148)
Phospho-EGFR (Y1173)
Phospho-EGFR (Y992)
Phospho-eIF4G (S1108)
Phospho-eNOS (S1177)
Phospho-eNOS/NOS III (S116)
Phospho-ErbB2/HER2 (Y1248)
Phospho-ERK 1/2 (T202/Y204)
Phospho-Estrogen Rec a (S118) (16JR)
Phospho-FADD (S194)
Phospho-FAK (Y397)
Phospho-FAK (Y576/577)
Phospho-FKHR (S256)
Phospho-FKHR (T24)/FKHRL1 (T32)
Phospho-GSK-3alpha/beta (Y279/216)
Phospho-Histone H3 (S10)
Phospho-IkappaB-alpha (S32/36) (5A5)
Phospho-IRS-1 (S612)
Phospho-Jak1 (Y1022/1023)
Phospho-MEK1/2 (S217/221)
Phospho-MSK1 (S360)
Phospho-mTOR (S2481)
Phospho-mtOR (S2448)
Musashi
Cleaved NOTCH
Phospho-NF-kappaB p65 (S536)
Phospho-p38 MAP Kinase (T180/Y182)
Phospho-p70 S6 Kinase (S371)
Phospho-p70 S6 Kinase (T389)
Phospho-p90RSK (S380)
Phospho-PDGF Receptor beta (Y716)
Phospho-PDGF Receptor beta (Y751)
Phospho-PKA C (T197)
Phospho-PKC alpha (S657)
Phospho-PKC zeta/lambda (T410/403)
Phospho-PKCdelta (T505)
Phospho-PKCtheta (T538)
Phospho-PRAS40 (T246)
Phospho-PTEN (S380)
Phospho-Pyk2 (Y402)
Phospho-Ras-GRF1 (S916)
Phospho-S6 Ribosomal Protein (S235/236) (2F9)
Phospho-SAPK/JNK (T183/Y185)
Phospho-Shc (Y317)
Phospho-Stat3 (Y705)
Phospho-Stat5 (Y694)
Phospho-VEGFR 2 (Y951)
Phospho-VEGFR 2 (Y996)
Smac/Diablo

Staining was performed using Catalyzed Signal Amplification System kit (Dako, Carpinteria, Calif.), and the stained images were acquired using NovaRay Image Acquisition Software (Alpha Innotech, San Leandro, Calif.). The images were analyzed using MicroVigene software (Vigenetech, Inc., Carlisle, Mass.), which identifies sample spots, subtracts local background, averages replicates and normalizes each sample for total protein. The data was then clustered and displayed as “heatmaps” of signaling profiles, as described in International Patent Application No. PCT/US09/044,903, which is incorporated herein by reference in its entirety. One heat map showed the unsupervised clustering analysis of the indicated target proteins in eight primary CRC tumors from patients that developed metastatic metachronous tumors compared to eight primary CRC tumors that did not progress to metastatic metachronous disease. The activated target proteins are listed in Table 1. A second heat map showed the indicated target proteins in 22 primary CRC tumors in patients with lymph node infiltration versus 22 primary CRC tumors without lymph node infiltration. The activated target proteins are listed in Table 2. A third heat map showed the indicated target proteins in the eight primary CRC tumors from patients that developed metastatic metachronous tumors compared to the fifty tumors that did not (14 lymph node positive, 36 non-metastatic). Likewise, cutpoints were determined using the methods described above to distinguish the activation status of each target.

The results are shown in Tables 2-4. Comparisons were made between the eight distant metachronous metastatic (MM) primary CRC samples and eight non-metastatic primary CRC samples. As shown in Table 2, the 23 statistically different “endpoints” (target proteins) show multiple activation changes in the EGFR and AKT/mTOR pathways between the MM CRC samples and the non-metastatic CRC samples.

TABLE 2
Activated signaling proteins in metastatic
versus non-metastatic CRC primary tumors.

| Endpoint | P. Value | Metastatic |
|---|---|---|
| Cl-Caspase9 | 0.018 | ↑ |
| Cox2 | 0.0003 | ↑ |
| EGFR | 0.004 | ↑ |
| pmTOR(S2481) | 0.054 | ↑ |
| EGFR(L858Mut) | 0.058 | ↑ |
| p4EBP1(S65) | 0.007 | ↑ |
| pAdducin(S662) | 0.047 | ↑ |
| pBAD(S136) | 0.07 | ↑ |
| pcAbl(T735) | 0.008 | ↑ |
| pcAbl(Y245) | 0.005 | ↑ |
| pcKit(Y703) | 0.012 | ↑ |
| pcRaf(S338)(56A6) | 0.003 | ↑ |
| pEGFR(Y1148) | 0.0002 | ↑ |
| pStat3(Y705) | 0.012 | ↑ |
| pHistoneH3(S10) | 0.046 | ↑ |
| plRS(S612) | 0.0002 | ↑ |
| Cl-NOTCH | 0.0002 | ↑ |
| pEGFR(Y1173) | 0.009 | ↑ |
| pPDGFRbeta(Y751) | 0.0002 | ↑ |
| pPyk2(Y402) | 0.018 | ↑ |
| pS6RibosomalProtein(S235/236) | 0.0006 | ↑ |
| pStat5(Y694) | 0.003 | ↑ |
| pVEGFR9Y951) | 0.035 | ↑ |

Likewise, Table 3 shows the statistically different signaling proteins for the lymph node positive CRC tumors versus lymph node negative CRC tumors.

TABLE 3
Activated signaling proteins in primary CRC tumors that
are lymph node positive versus those that are not.

| Endpoint | P. Value | Lympho+ |
|---|---|---|
| EGFR(L858Mut) | 0.01 | ↑ |
| p4EBP1(S65) | 0.003 | ↑ |
| pcAbl(Y245) | 0.008 | ↑ |
| pChk2(S33/35) | 0.046 | ↑ |
| pcRaf(S338)(56A6) | 0.026 | ↑ |
| pEGFR(Y1148) | 0.011 | ↑ |
| pGSK3alpha/beta(Y279/216) | 0.045 | ↓ |
| CI-NOTCH | 0.047 | ↑ |
| pPDGFRbeta(Y751) | 0.0006 | ↑ |
| pPKCalpha(S657) | 0.03 | ↓ |
| pS6RibosomalProtein(S235/236) | 0.011 | ↑ |

Furthermore, signaling differences in the patient-matched epithelium and stromal cell isolates reveal that different cell types within the tumor could present specific and characteristic phosphoproteomic profiles (data not shown).

These results indicate that the primary tumors from patients with occult distant metastases have a statistically significant elevation in the activation of many signaling proteins in the growth factor receptor (e.g., PDGFR, VEGFR, c-Kit EGFR) pathways. These pathways appear to link downstream with the mTOR pathway. Interestingly, AKT itself did not appear to be differentially phosphorylated in these samples. The differentially activated signaling proteins discovered in this study are all involved in cell proliferation and migration and may be involved in the dissemination of the primary lesion.

### Example 2

The tumors from Example 1 were further characterized to develop prognostic markers for disease progression. The eight primary tumors from patients that developed metachronous metastases were compared to the fifty tumors from patients that did not (14 with lymph node infiltration, 36 without). The results were analyzed using unsupervised clustering, and the results were shown in the third heat map, which showed the indicated target proteins in the eight primary CRC tumors from patients that developed metastatic metachronous tumors compared to the fifty tumors that did not (14 lymph node positive, 36 non-metastatic). The numerical data are provided in Table 4.

TABLE 4

| | | Regulation in | | | | |
|---|---|---|---|---|---|---|
| | | patients with | | | AUC Pathway | AUC Pathway |
| Target | P value | occult metastasis | AUC (8 vs 50) | AUC (8vs14) | Score (8vs50) | Score (8vs14) |
| CI-Caspase9 D315 | 0.0163 | + | 0.7688 | 0.7589 | 0.8214 | 0.8725 |
| CI-NOTCH V1744 | 0.0003 | + | 0.9063 | 0.8973 | | |
| EGFR | 0.0021 | + | 0.8425 | 0.8661 | | |
| p4EBP1 S65 | 0.0130 | + | 0.7613 | 06161 | | |
| pAbl T735 | 0.0075 | + | 0.7975 | 0875 | | |
| pAbl Y245 | 0.0008 | + | 0.8738 | 0.7857 | | |
| pBAD S136 | 0.0033 | + | 0.8276 | 0.8661 | | |
| pcKit Y703 | 0.0003 | + | 0.9000 | 0.9286 | | |
| pEGFR Y1148 | 0.0006 | + | 0.8713 | 0.7679 | | |
| pmTOR S2481 | 0.0279 | + | 0.7450 | 07589 | | |
| pp70 S6 S371 | 0.0185 | + | 0.7625 | 0.7589 | | |
| pPKCa S657 | 0.0485 | − | 0.7200 | 0.6607 | | |
| pPDGFRβ Y751 | 0.0001 | + | 0.9275 | 0.8839 | | |
| pPyk2 Y402 | 0.0010 | + | 0.8476 | 0.9107 | | |
| pSTAT5 Y694 | 0.0040 | + | 0.8200 | 0.7857 | | |
| pVEGFR Y951 | 0.0391 | + | 0.7313 | 0.6696 | | |
| Cox2 | <0.0001 | + | 0.9475 | 0.9286 | | |
| pAdducin S662 | 0.0012 | + | 0.8600 | 0.9196 | | |
| pBcl2 S70 | 0.0152 | + | 0.7425 | 0.6786 | | |
| pEGFR Y1173 | 0.0073 | + | 0.8025 | 0.9107 | | |
| pERK 1/2 T202/Y204 | 0.0127 | + | 0.7738 | 0.8214 | | |
| pHistone-H3 S10 | 0.0149 | + | 0.7713 | 0.8482 | | |
| pIRS S612 | 0.0004 | + | 0.8975 | 0.9464 | | |
| pcRaf S338 | 0.0002 | + | 0.9150 | 0.8929 | | |
| pS6 Ribosomal Protein | 0.0010 | + | 0.8538 | 0.7589 | | |
| S235-236 | | | | | | |

The targets that are most closely associated with the development of metastases (p value <0.01) are provided in Table 5.

TABLE 5
Target proteins in primary CRC tumors with best prognostic value.

| Target | Activation type |
|---|---|
| Cox2 | Increase in total Cox2 protein |
| pBAD S136 | Phosphorylation |
| pcKit Y703 | Phosphorylation |
| pPDGFRb Y751 | Phosphorylation |
| pEGFR Y1173 | Phosphorylation |
| pS6RibProt S235/S236 | Phosphorylation |
| pmTOR S2481 | Phosphorylation |
| pAbl T735 | Phosphorylation |
| pAdducin S662 | Phosphorylation |
| pBcl2 S70 | Phosphorylation |
| pcRaf S338 | Phosphorylation |
| pEGFR Y1148 | Phosphorylation |
| Cl-NOTCH | Proteolytic cleavage |

Interestingly, pPKC alpha is activated only in primary CRC tumors from patients that did not develop metastases.

To develop a high specificity prognostic test, a pathway signature score was calculated. The relative intensity of each of the target proteins in Table 4 were summed, then the sum was divided by the relative intensity of the target protein that is activated only in non-metastatic tumors, pPKC alpha. The pathway signature score from each sample was plotted in FIG. 1, grouped according to whether the tumor came from a patient that developed metastatic metachronous tumors (MET), had no metastases but did have lymph node infiltration (L+), or no metastases and no lymph node infiltration (NON-MET). A cutpoint was placed just below the lowest score for the MET samples, at value 15. Samples with scores above this cutpoint are considered at risk for developing metastases, and samples with scores below this value were considered to be non-metastatic.

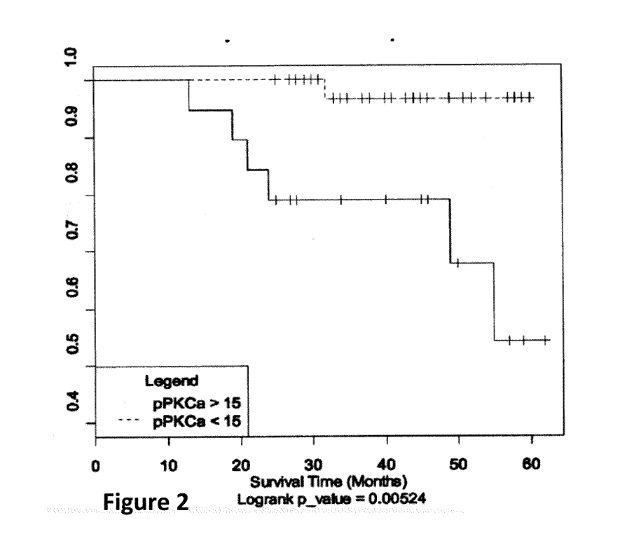

To test the correlation between the pathway signature score of FIG. 1, the patients were followed for five years post-surgery to generate the Kaplan-Meir survival plot shown in FIG. 2. The patients were grouped according to their pathway signature score, with those above the cutpoint value of 15 shown in the bottom line, and those with scores below the cutpoint shown in the upper line. With a greater than 95% survival rate in the low score population, versus a less than 60% survival rate in the high score population, the usefulness of using the pathway signature score to distinguish CRC patients with high and low risk of metastases was confirmed.

1.-38. (canceled)

39. A method for predicting if a subject with colorectal cancer is likely to develop one or more metastases or has occult metastasis, comprising the steps of:
(A) measuring the activation level of one or more target proteins in a sample from the subject's primary tumor, wherein the one or more target proteins are selected from the group consisting of:
a. mTOR,
b. 4EBP1,
c. Adducin,
d. cKit,
e. cRaf,
f. Stat3,
g. HistoneH3,
h. IRS,
i. PDGFR beta,
j. Pyk2,
k. S6 Ribosomal Protein,
l. Stat5,
m. VEGFR,
n. C1-Caspase9,
o. C1-NOTCH,
p. Cox2,
q. EGFR,
r. pBAD,
s. pcAb1, and
t. pPKC alpha; and
(B) comparing the activation level of (A) to positive and/or negative reference standards to determine if the target protein is activated;
wherein the activation level of (A) is determined by measuring the phosphorylation of the target protein, the total amount of the target protein or the proteolytic cleavage products of a target protein; and
wherein the activation of one or more target proteins indicates that the patient is likely to likely to develop metastases.

40. The method of claim 39, further comprising
(c) calculating a pathway signature score by
(i) summing the activation levels of the target proteins a.-s. of (A); and
(ii) dividing the sum of (i) by the activation level of a target protein associated with non-metastases, and
(D) determining a cutpoint of the pathway signature score of (C) such that none of the subjects with samples having a pathway signature score below the cutpoint develop metastases.

41. The method of claim 39, wherein the subject is a human patient and the colorectal cancer is likely to metastasize to the patient's liver.

42. The method of claim 39, wherein the sample is prepared by the steps comprising:
(i) isolating epithelial cells from the sample;
(ii) lysing the epithelial cells to form a lysate; and
(iii) contacting the lysate with a detectable label to detect the target protein.

43. The method of claim 39, wherein step (A) comprises measuring the level of phosphorylation of one or more of the following proteins:
a. pCox2,
b. pBAD,
c. pcKit,
d. pPDGFRb,
e. pEGFR,
f. pS6 Ribosomal protein,
g. pmTOR,
h. pAb1,
i. pAdducin,
j. pBcl2,
k. pcRaf,
l. pEGFR,
m. C1-NOTCH, and
n. PKC alpha.

44. The method of claim 39, wherein the activation levels of at least two of the proteins are measured.

45. The method of claim 39, wherein the target proteins of (A) are at least one of mTOR, cKit, PDGFR beta, EGFR, Cox2 and VEGFR.

46. A method for treating, delaying or preventing metastasis in a human patient with colorectal cancer comprising the steps of:
(A) measuring the activation level of one or more target proteins in a sample from the patient's primary tumor, wherein the one or more target proteins are selected from the group consisting of:
a. mTOR,
b. 4EBP1,
c. Adducin,
d. cKit,
e. cRaf,
f. Stat3,
g. HistoneH3,
h. IRS,
i. PDGFR beta,
j. Pyk2,
k. S6 Ribosomal Protein,
l. Stat5,
m. VEGFR,
n. C1-Caspase9,
o. C1-NOTCH,
p. Cox2,
q. EGFR,
r. pBAD,
s. pcAb1, and
t. PKC alpha; and
(B) comparing the activation level of (A) to positive and/or negative reference standards to determine if the target protein is activated; and
(C) treating the patient with a targeted or aggressive therapy if the activation of one or more target proteins of (B) indicates that the patient is likely to develop metastases,
wherein the activation level of (A) is determined by measuring the phosphorylation of the target protein, the total amount of the target protein or the proteolytic cleavage products of a target protein.

47. The method of claim 46, wherein step (A) comprises measuring the level of phosphorylation of one or more of the following proteins:
a. pCox2,
b. pBAD,
c. pcKit,
d. pPDGFRb,
e. pEGFR,
f. pS6 Ribosomal protein,
g. pmTOR,
h. pAb1,
i. pAdducin,
j. pBc12,
k. pcRaf,
l. pEGFR,
m. C1-NOTCH, and
n. PKC alpha.

48. The method of claim 46, wherein step (C) comprises treating the patient with an effective amount of a therapeutic agent that targets at least one of the activated target proteins.

49. The method of claim 48, wherein the therapeutic agent is one or more agents selected from the group consisting of CELECOXIB, REFECOXIB, TORISEL, TARCEVA, LAPATINIB, IRESSA, ERBITUX, BEVTUZIMAB, AVASTIN, GLEEVEC, DASATINIB, and SUTENT.

50. The method of claim 46, further comprising administering a conventional chemotherapeutic agent to the patient.

51. A kit for selecting a treatment for a subject having CRC comprising:
(A) one or more reagents for determining the activation level of one or more target proteins in a sample from the subject's primary tumor, wherein the one or more target proteins are selected from the group consisting of:
a. mTOR,
b. 4EBP1,
c. Adducin,
d. cKit,
e. cRaf,
f. Stat3,
g. HistoneH3,
h. IRS,
i. PDGFR beta,
j. Pyk2,
k. S6Ribosomal Protein,
l. Stat5,
m. VEGFR,
n. C1-Caspase9,
o. C1-NOTCH,
p. Cox2,
q. EGFR,
r. pBAD,
s. pcAb1, and
t. PKC alpha; and
(B) instructions for performing the assay.

52. The kit of claim 51, comprising reagents for assaying the phosphorylation state of at least one of mTOR, cKit, PDGFR beta, EGFR, Cox2 and VEGFR.

53. The kit of claim 51, wherein the activation level is determined by measuring the level of phosphorylation of one or more of the target proteins.

54. The kit of claim 51, wherein the sample is prepared by the steps comprising:
(i) isolating epithelial cells from the sample;
(ii) lysing the epithelial cells to form a lysate; and
(iii) contacting the lysate with a detectable label to detect the target protein.

55. A method of using the kit of claim 51 to select a treatment for a human with CRC, comprising the steps of:
(A) measuring the activation level of one or more of the target proteins in the sample, wherein the activation level is determined by measuring the phosphorylation of the target protein, the total amount of the target protein, or the proteolytic cleavage products of the target protein;
(B) comparing the activation level of the one or more target proteins to positive and/or negative reference standards to determine if the target protein is activated; and
(C) treating the patient with a targeted or aggressive therapy, if one or more of the target proteins are activated.

56. The method of claim 55, wherein the sample is prepared by the steps comprising:
(i) isolating epithelial cells from the sample;
(ii) lysing the epithelial cells to form a lysate; and
(iii) contacting the lysate with a detectable label to detect the target protein.

57. A pharmaceutical composition, comprising a therapeutically effective amount of:
(A) a targeted therapeutic agent of at least two target proteins selected from the group consisting of mTOR, cKit, PDGFR, EGFR, Cox2 and VEGFR; and
(B) a pharmaceutically acceptable carrier.

58. The pharmaceutical composition of claim 57, further comprising a therapeutically effective amount of carboxyamido imidazole.

59. The pharmaceutical composition of claim 57, wherein the targeted therapeutic agent is one or more agents selected from the group consisting of CELECOXIB, REFECOXIB, TORISEL, TARCEVA, LAPATINIB, IRESSA, ERBITUX, BEVTUZIMAB, AVASTIN, GLEEVEC, DASATINIB, and SUTENT.
